(19) United States
(12) Patent Application Publication
(10) Pub. No.: US 2011/0172675 A1
(43) Pub. Date: Jul. 14, 2011

(54) OCULAR INLAY DELIVERY SYSTEM AND METHOD OF USE

(75) Inventors: Randall C. Danta, Tustin, CA (US); Kyle Webb, Carlsbad, CA (US)

(73) Assignee: ACUFOCUS, INC., Irvine, CA (US)

(21) Appl. No.: 13/005,140

(22) Filed: Jan. 12, 2011

Related U.S. Application Data
(60) Provisional application No. 61/294,393, filed on Jan. 12, 2010.
(60) Provisional application No. 61/308,265, filed on Feb. 25, 2010.

(51) Int. Cl. A61F 9/007

(52) U.S. Cl. 606/107

(57) ABSTRACT

The present application describes an ocular inlay delivery system that can include a loader, forceps, and tray system. The loader can be configured to receive an ocular inlay, protect it during handling and shipment, and present it for removal during surgery, receive jaws of surgical forceps and align the jaws in relation to the inlay allowing the forceps to grip the inlay in a proper orientation. Forceps can be configured to fit within the channel of the loader, grip the inlay using a clamping motion, maintain a grip on the inlay during transfer to the eye of a patient, deposit the inlay in the eye of a patient, and release the inlay using a sliding motion. The tray can be configured to restrict movement of the loader mechanism during shipment and handling.

### CROSS-REFERENCE TO RELATED APPLICATIONS

This application claims the benefit of U.S. Provisional Application Nos. 61/294,393, filed Jan. 12, 2010 and 61/308,265, filed Feb. 25, 2010, the entirety of each of which is hereby incorporated by reference.

### BACKGROUND

1. Field of the Invention

This application relates to methods and apparatuses for delivering ocular implants (e.g., corneal inlays).

2. Description of the Related Art

Standard techniques for manipulating and storing corneal inlays involve the use of typical tweezers and containers. While typical tweezers provide ability to manipulate corneal inlays, obtaining a grip on the corneal inlay with typical tweezers can be difficult. Furthermore, the corneal inlay must be placed between corneal layers in proper alignment with the patient's vision, and typical tweezers generally do not provide the enhanced precision needed for proper placement.

The use of typical containers as packaging for corneal inlays can create further difficulty in manipulating corneal inlays. Typical containers have a volume much larger than the corneal inlay. The larger volume allows the corneal inlay to move freely inside the container, making it difficult to locate and grip the corneal inlay at the time of surgery.

### SUMMARY

Placing a corneal inlay within the cornea of an eye requires significant skill and dexterity because the inlay can be relatively small and lightweight. Manipulating a corneal inlay with standard techniques and tools can be difficult. Therefore, there is a need for tools and techniques that can more easily manipulate and control placement of a corneal inlay, such as an inlay that includes a small aperture, but can also be beneficial for any inlay that is designed to be positioned within a cornea.

In certain embodiments, a loader for use with a corneal inlay is provided. The loader can include a platform having a recess configured to receive a corneal inlay, and a cover member slideably contacting the platform. In a closed position, the cover member at least partially covers the recess, and in an open position, the cover member substantially does not cover the recess. In some embodiments, the recess has a diameter substantially the same as the corneal inlay.

The platform may further include a channel extending from the recess to an exposed surface of the platform and configured to receive a handling tool. The loader can include a retainer within the recess configured to restrict the corneal inlay from moving to the bottom of the recess. The retainer may have a shelf between a top and a bottom of the recess, and a first portion of the recess between the shelf and the bottom of the recess can be configured to receive the handling tool.

The cover member may further include a retaining feature that extends at least partially into the recess of the platform when in the closed position to prevent the corneal inlay from escaping from the recess. The platform can include one or more slots and the retaining feature of the cover member can include one or more rails that are receivable into the one or more slots in the platform.

In certain embodiments, a tray for use with the loader is provided. The tray can include a locking mechanism, wherein in a locked position, the locking mechanism restricts the cover member from moving into the open position, and in an unlocked position, the locking mechanism allows the cover member to move into the open position. The locking mechanism can include a protrusion on a surface of the tray, wherein in a locked position, the protrusion extends at least partially into an opening of the loader, and in an unlocked position, the protrusion does not extend into the opening of the loader.

In certain embodiments, an inlay handling tool for gripping a corneal inlay is provided. The inlay handling tool can include a first arm member having a first jaw member disposed at a distal end thereof, and a second arm member having a second jaw member disposed at a distal end thereof. The first arm member and second arm member are coupled together to provide vertical separation and horizontal translation of the first jaw member and the second jaw member relative to one another.

The inlay handling tool may further include pins on opposite sides along a length of the first arm member, and slots on opposite sides along a length of the second arm member. The pins of the first arm member are receivable within the slots of the second arm member to movably couple the first arm member and the second arm member together, the second jaw member being able to at least partially rotate relative to the first jaw member about the pins to provide the vertical separation, and the pins of the first arm member being able to move within the slot of the second arm member to provide the horizontal translation of the second jaw member relative to the first jaw member.

The slot of the second arm member may further include an opening that the pins may pass through so that the first arm member and second arm member can be separated. The first arm member and the second arm member may biased together. For example, the first arm member can include a cantilever spring disposed at a proximal end thereof, the second arm member further comprises a platen disposed at a proximal end thereof, and wherein the cantilever spring provides a force on the platen to bias the first jaw member and the second jaw member toward one another. In some embodiments, the first jaw member includes a ring and the second jaw member includes a partial ring.

In certain embodiments, a method of using a corneal inlay delivery system is provided. The method can include providing a loader having a corneal inlay disposed therein, wherein the loader comprises a surface for supporting the corneal inlay and a cover member at least partially covering the corneal inlay. The method can further include sliding the cover member to an open position, inserting a portion of an inlay handling tool beneath the inlay, gripping the corneal inlay with the inlay handling tool, and removing the corneal inlay from the loader.

The method may further include disengaging a cover member lock that prevents the cover member from sliding into the open position and/or inserting the inlay handling tool through a channel extending from the recess to an exposed surface of the platform.

In certain embodiments, a method of using an inlay handling tool to implant a corneal inlay into a cornea of a patient is provided. This method may be separate or combined with the method of using a corneal inlay delivery system. The method of using an inlay handling tool can include inserting at least a portion of the inlay handling tool with the corneal inlay into a pocket in, or under a flap of, a cornea of a patient, horizontally traversing a jaw member of the inlay handling tool to expose a side of the corneal inlay, adhering the exposed side of the corneal inlay to an internal surface of the cornea, and removing the corneal inlay handling tool from the corneal surface.

### BRIEF DESCRIPTION OF THE DRAWINGS

FIG. 1A is a perspective view of an embodiment of a loader;

FIG. 1B is a top exploded perspective view of the loader of FIG. 1;

FIG. 1C is a bottom exploded perspective view of the loader of FIG. 1;

FIG. 2 is a perspective view of the loader of FIG. 1 in an open configuration with an embodiment of a mask positioned above the loader;

FIG. 3 is a cross-sectional view through 3-3 of FIG. 1;

FIG. 4 is a perspective view of an embodiment of forceps positioned to grasp an inlay from the loader of FIG. 2;

FIG. 5A illustrates the forceps of FIG. 4 grasping the inlay for transfer to a patient;

FIG. 5B illustrates the forceps of FIG. 4 placing the inlay into a pocket in the cornea of a patient;

FIG. 6A is an exploded perspective view of the loader of FIG. 1, an embodiment of a first tray, and an embodiment of a second tray;

FIG. 6B is a side sectional view through 6B-6B of FIG. 6A of the loader mated with the first tray;

FIG. 6C is a side section view through 6B-6B of FIG. 6A of the loader not mated with the first tray so that the loader can be in an opened configuration;

FIG. 7A is a perspective view of an embodiment of forceps in a closed position;

FIG. 7B is a perspective view of the forceps of FIG. 7A with one of the jaw members slid back;

FIG. 8A is a top view of an embodiment of a jaw member of an inlay handling tool with a pear shape;

FIG. 8B is a side view of the jaw member of FIG. 8A;

FIG. 9A is a perspective view of an embodiment of forceps with a pear shape distal end;

FIG. 9B is a perspective view of an embodiment of forceps with a round distal end;

FIG. 10A is a perspective view of an embodiment of a jaw member of an inlay handling tool with beveled edges; and

FIG. 10B is a side view of the jaw member of FIG. 10A.

### DETAILED DESCRIPTION

Thin corneal inlays, such as corneal inlays described in U.S. Pat. No. 7,628,810 and International Application No. PCT/US2010/045541, each of which is hereby incorporated by reference in its entirety, can range in thickness from approximately 4 to 10 microns, making them very delicate. The fragile nature of the device demands precise handling to avoid tearing, wrinkling, or folding. Challenges are encountered in protecting such a thin inlay during various stages of manufacture, storage, transportation, and handling, while presenting it in an available condition for use in the surgical environment.

One embodiment of a corneal inlay package includes a tissue capsule approximately 1″ in diameter and ¼″ high. While marginally successful, such packages have proven difficult to use. During manufacture, the inlay is placed into the capsule using a vacuum pick. During shipment, the inlay may move around inside the capsule. At the time of surgery, the 3.8 mm diameter inlay may be found anywhere within in the capsule including adhering to the lid or sides, or lying on the floor.

A difficulty with the above embodiment is picking up the inlay and orienting it for successful insertion into the eye of a patient. The surgeon or an assistant opens the capsule, finds the inlay, works a jaw of the forceps under an edge of the inlay, captures the inlay by an edge, and lifts it from the surface of the capsule. Once the inlay has been captured, it may be placed into a well of a loading block. The loading block can be an additional component configured for the purpose of temporarily holding the inlay. Once constrained in the loading block, forceps may then be re-positioned to align with the center of the inlay, or other appropriate grip point as dictated by the shape of the loading dock and the jaws of the forceps.

A further difficulty of the above embodiment is maintaining an appropriate grip on the inlay while transporting it from the packaging to the patient. Too light a grip and the inlay may slip and become lost. Too hard a grip and the inlay may become deformed. If the forceps jaws are ribbed or textured for a secure grip, the inlay may take on the pattern.

A further problem of the above embodiment is to deposit the inlay successfully in the eye of a patient. Current corneal surgery practice uses either a flap or a pocket. In flap surgery, an outer layer of the cornea is hinged open exposing the stromal bed. Depositing an inlay onto the stromal bed includes simply placing the inlay onto the bed, dislodging the inlay from the jaws of the forceps, performing any fine positional adjustments, then closing the flap.

In pocket surgery, the inlay is inserted between closely spaced upper and lower surfaces of a corneal pocket. One difficulty is lifting the edge of the pocket to gain entry. The forceps can be thin and/or have a protruding edge to get the opening started. However, these same features may represent abrasion or cut hazards later in the procedure. Using a second tool to open the pocket is possible, but such a technique has not received wide acceptance.

Once the pocket is open by inserting the edge of the forceps into the opening, the tip or tips of the forceps may be further inserted into the pocket until the inlay resides generally over the center of the pupil, e.g., by inserting approximately 7 mm into the pocket. Preferably, the jaws of the forceps cover a sufficient portion of the inlay to complete the insertion of without folds or wrinkles forming along the sides of the inlay.

Fine positioning of the inlay may then be undertaken. Preferably, the jaws continue to grip the inlay and support its movement in, out, left, or right without folding any of the edges.

Finally, the jaws are released and the forceps withdrawn, leaving the inlay in its designated location. Preferably, the jaws of the forceps are separated from the inlay. This may prove difficult both due to limited space and adhesion between the inlay and the jaws of the forceps. If the forceps use a hinged opening motion, the amount of separation is limited by the ability of the corneal tissue to separate. Once open, there is the further problem of freeing the inlay from the jaws of the forceps and inducing it to adhere instead to the surrounding tissue. In certain embodiments, small wings or overhangs of inlay protruding from both sides of the forceps jaws may stick to the corneal tissue causing the inlay to dislodge from the jaws. Preferably, the small wings are limited to what can be safely exposed without folding of the edges. Upon separating the jaws, there may be little material in contact with corneal tissue to induce the inlay to remain behind as the forceps are withdrawn.

It should become apparent that even with surgical magnification and the high manual dexterity of today's ophthalmic surgeon, the above tasks are difficult to perform reliably with existing tools and methods. An object of the present application is to provide a system and method to overcome the above difficulties in storing, transporting, and successfully inserting a thin corneal inlay in the eye of a patient.

### Loader Embodiments

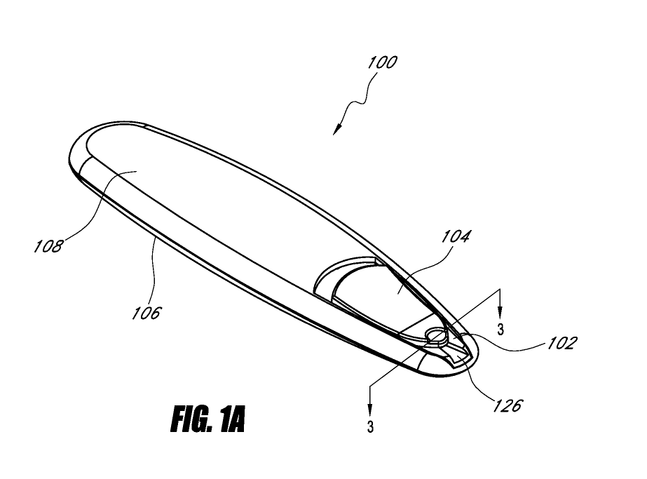

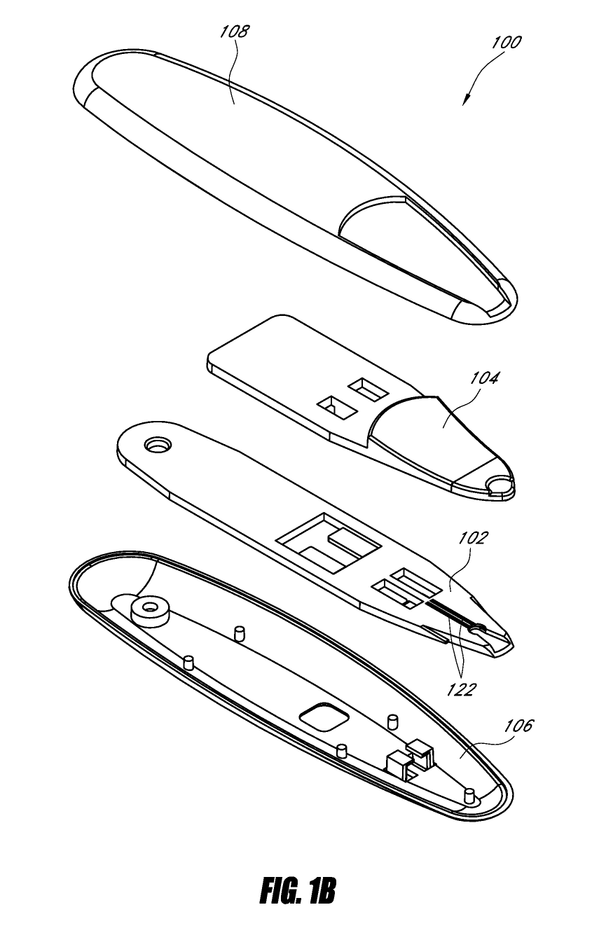

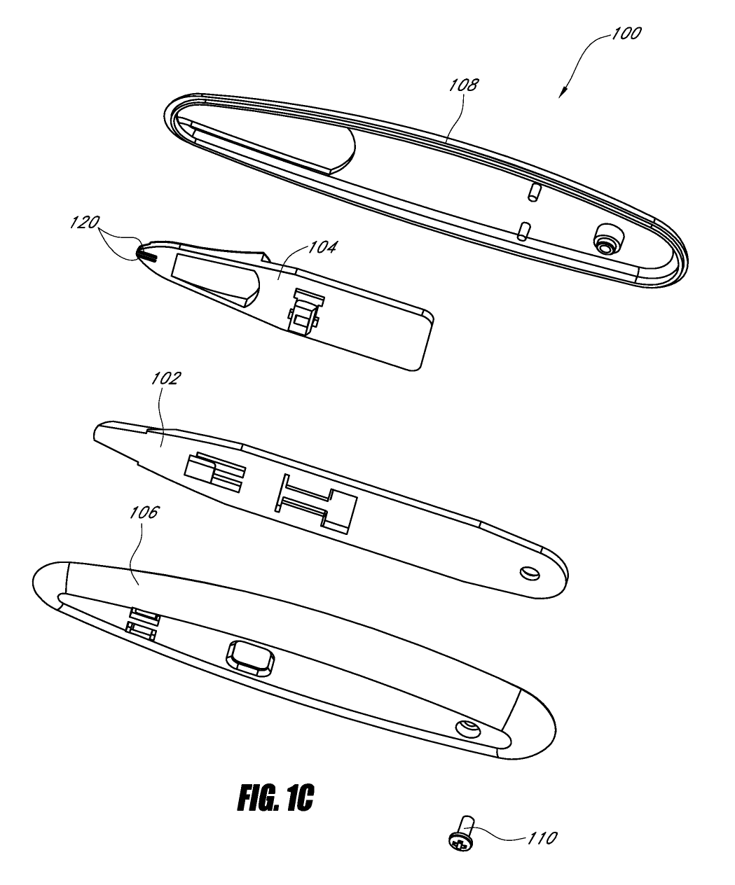

A first component of a corneal inlay delivery system, a loader, can include a platform and a cover member. One embodiment of a loader 100 is illustrated in FIGS. 1A-C. FIG. 1A is a top perspective view, FIG. 1B is a top exploded perspective view, and FIG. 1C is a bottom exploded perspective view of the loader 100 which includes a platform 102 and a cover member 104. In certain embodiments, the cover member 104 is a slider. The loader 100 can further include a base 106, a cover 108 and a way or a means to hold the components of the loader 100 together. For example, a screw 110 could be used to hold together the components. The cover 108 and base 106 can include textured external surfaces for gripping the loader 100 with a gloved hand. The cover 108 and base 106 may also provide color coding to distinguish product models, or simply to provide an aesthetically pleasing package. The exterior surfaces may be imprinted with logos or other product identification.

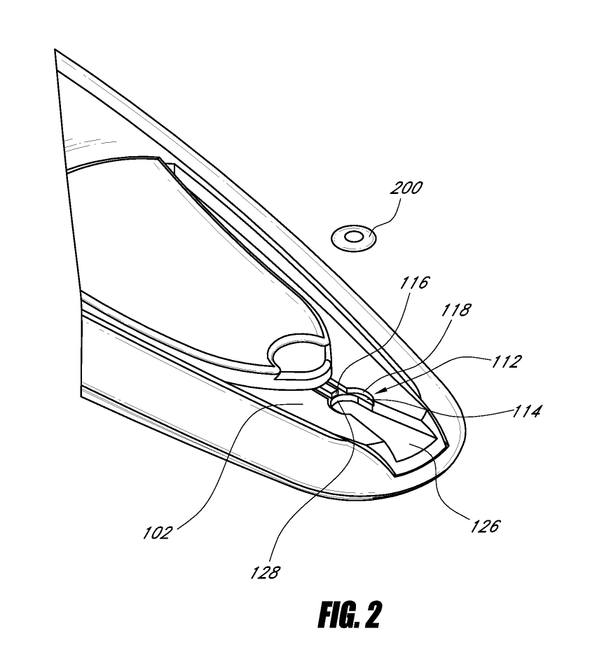

The platform 102 can serve as an internal component, and can be configured to receive an inlay 200 in a well or recess 112, as illustrated in FIG. 2. For example, the recess can have substantially the same shape, size, diameter, etc. as the inlay 200. In FIG. 2, the inlay 200 is positioned outside of the well 112 so that features of the well 112 can be seen. The well 112 can further include a shelf 114 to support at least some of the edges of the inlay 200 to constrain it from further movement in the downward direction. A raised rear wall 116 of the well 112 can provide a backstop and thus prevent the inlay 200 from movement in the rearward direction. Side walls 118 of the well can be circular in shape. In certain embodiments, the side walls 118 sweep out a perimeter of approximately 270 degrees. In other embodiments, the side walls 118 sweep out at least greater than 180 degrees. The extent to which the walls extend past 180 degrees provides material to constrain the inlay 200 from movement in the forward direction.

A cover member or slider 104 can be configured to move slideably between an open and a closed position. In the open position as illustrated in FIG. 2, the opening of the well 112 is available to receive an inlay 200, as during manufacture. In a closed position as illustrated in FIG. 1A, the slider 104 covers at least a portion of the well 112 and the inlay 200 when an inlay is present in the well 112. The slider 104 constrains the inlay 200 from moving in an upward direction.

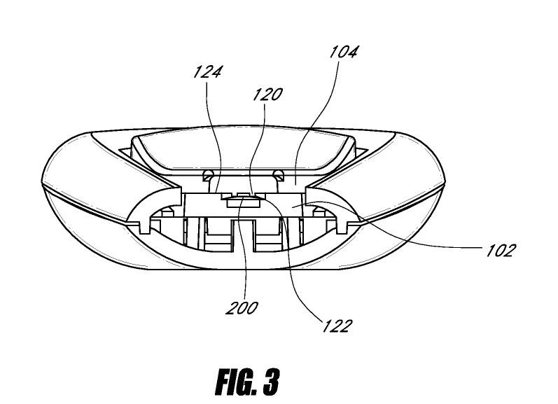

The slider 104 can be further configured with horizontal rails 120 (as illustrated in FIG. 1C) that fit into grooves 122 in the platform 102 (as illustrated in FIG. 1B). As illustrated in FIG. 3 which is a cross-sectional view through 3-3 of FIG. 1A, the rails 120 can extend below the surface of the platform 102 to further constrain the inlay 200 and prevent the inlay 200 from escaping between any possible gap 124 between the platform 102 and slider 104. The combination of platform 102 and slider 104 can protect and secure the inlay 200 during handling and shipment.

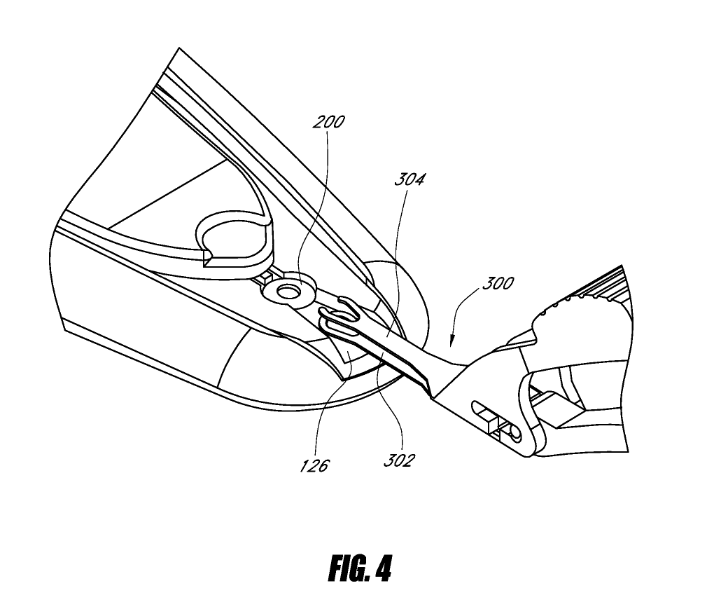

An important aspect of the loader 100 can be its ability to locate the inlay 200 in a known location for removal during surgery. This can be accomplished by providing a channel 126 configured to receive the jaws of forceps 300, as illustrated in FIG. 4. In a preferred embodiment, the channel 126 is sized to closely match the width of the jaws of the forceps 300.

In certain embodiments, when fully inserted into the loader 100, at least one of the jaws of the forceps 300 comes to rest against a backstop 128 (the backstop is shown in FIG. 2). The channel width and location of the backstop 128 can determine the position of the jaws of the forceps 300 relative to the position of the inlay 200. In addition, the inlay 200 can be properly orientated in the loader 100 so that the inlay 200 is properly orientated in the jaws of the forceps 300. Proper orientation of the inlay 200 in the jaws of the forceps can be important when the inlay 200 requires proper orientation when implanted in the cornea so that the posterior side of the inlay 200 is orientated posterior and the anterior side of the inlay 200 is orientated anterior.

At the time of surgery, the forceps 300 are opened by a hinging motion and the lower jaw 302 is slid into the channel 126 below the inlay 200, while the upper jaw 304 hovers above the inlay 200. The jaws may then be closed using a hinge motion thus capturing the inlay 200 securely between the jaws of the forceps 300.

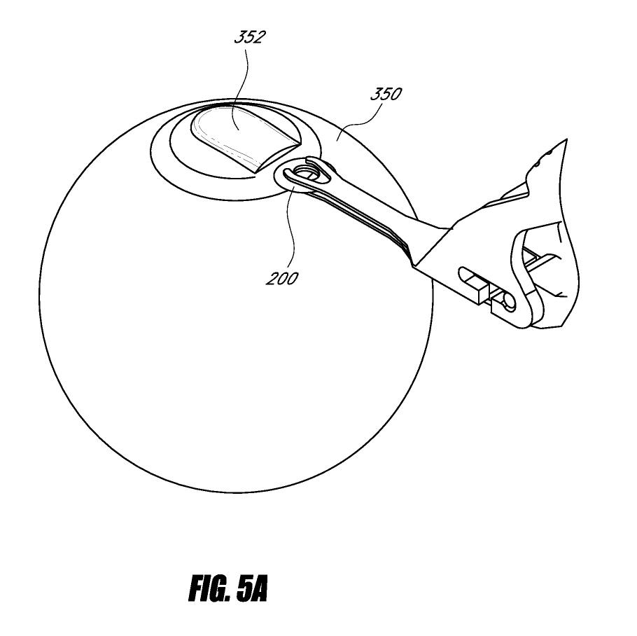

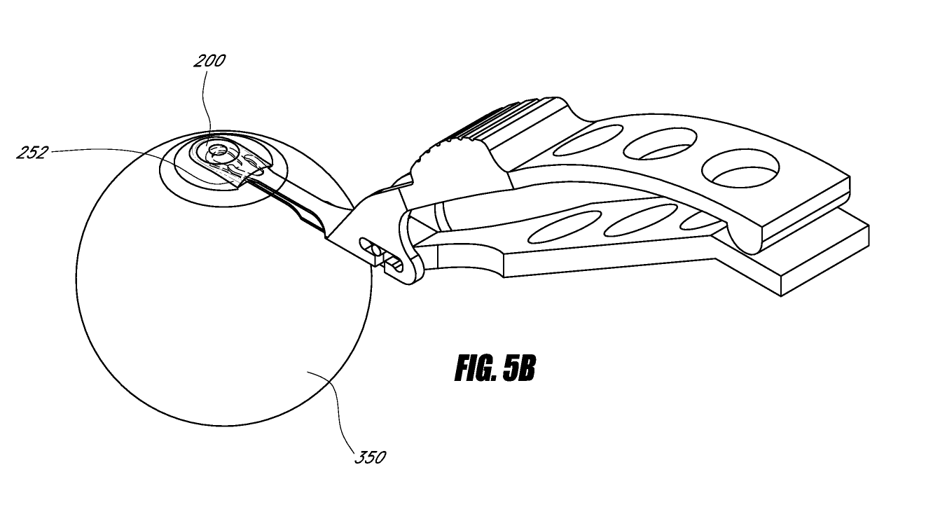

The inlay 200 can then be removed from the loader 100 as the jaws of the forceps 300 are held against the inlay 200. The jaws of the forceps 300 are then held closed while being transferred to the operating table. The surgeon may perform the entire operation, or an assistant may obtain the inlay 200 from the loader 100 and then hand the forceps 300 to the surgeon. Either way, the surgeon ultimately holds the forceps 300 containing the inlay 200 in preparation for insertion into the eye 350 of a patient, as illustrated in FIG. 5A.

As illustrated in FIG. 5B, the inlay 200 can then be deposited in the eye 350 of a patient, which has previously been prepared with either a pocket or flap 352. The inlay 200 can then be released and the forceps 300 can be removed from the eye 350 of the patient, as described below.

### Inlay Package Embodiments

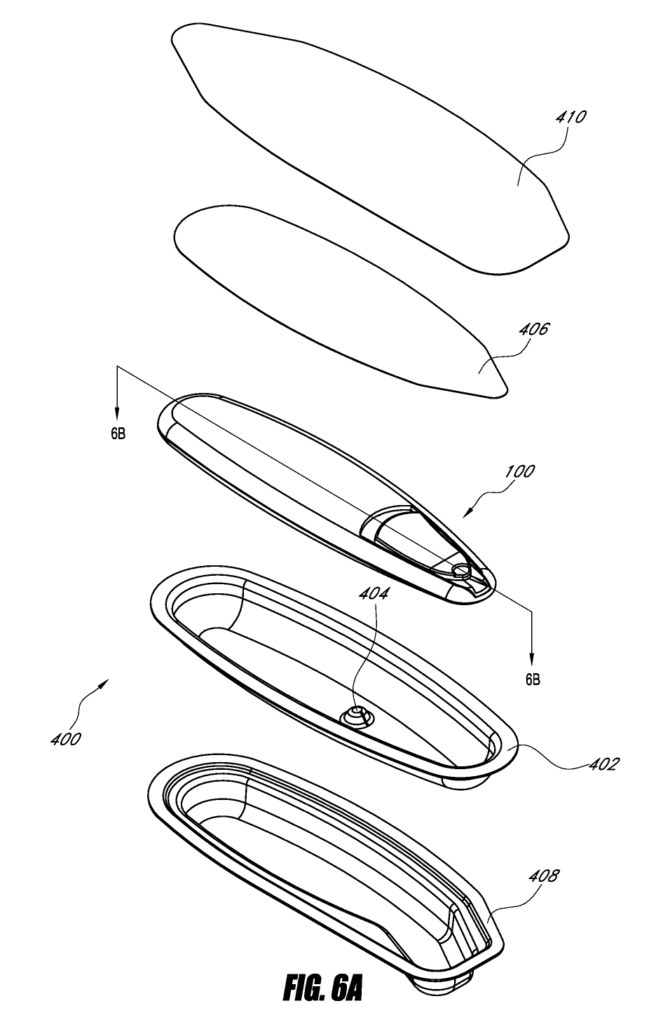

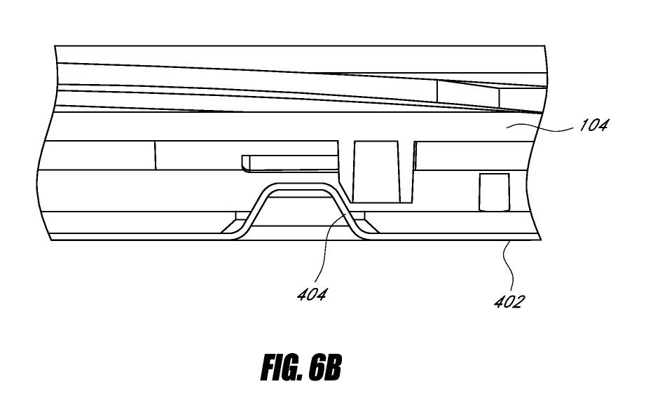

As illustrated in FIG. 6A, a second component of the corneal inlay delivery system can include a package 400, such as a tray. Once the loader 100 is populated with an inlay 200, the loader 100 can then be placed into a first tray 402 configured to protect the loader 100, to provide a sterile barrier, and to provide a key protrusion 404 that fits inside the loader 100 to ensure the slider 104 does not open during shipment. In certain embodiments, the first tray 402 is configured to prevent or restrict the slider 104 from moving into an open position. For example, the first tray 402 can include a locking mechanism that restricts the slider 104 from moving into an open position. FIG. 6B illustrates a cross-sectional view through 6B-6B of FIG. 6A that illustrates the key protrusion 404 of the first tray 402 that protrudes into the loader 100 to restrict the slider 104 from moving into the open position (e.g., keeps the slider 104 in the closed position). For comparison, FIG. 6C illustrates the loader 100 without the package 400 wherein the slider 104 is in an open position.

The first tray 402 can then be sealed using a sterile barrier 406 such as Tyvek® or other material. The first tray 402 can also be placed within a second tray 408, and the second tray 408 can be sealed with a second sterile barrier 410 in a similar fashion. The double tray can be placed into appropriate carton along with instructional and identification materials and sent to the sterilizer.

The loader 100 itself may also be configured so that in one configuration the slider 104 is restricted moving into the open position while in another configuration the slider 104 can moved into the open position. For example, the loader 100 may include a locking mechanism for the slider 104. The locking mechanism of the loader may be used in conjunction with the first tray's 402 locking mechanism or alternatively to the first tray's 402 locking mechanism.

### Handling Tool Embodiments

An inlay handling tool can be a third component of the corneal inlay delivery system. In use, the handling tool can serve the function of grasping the inlay, lifting it out of the well, transferring it to the patient, and inserting the inlay into the eye of a patient. In certain embodiments, the construction of the handling tool allows the user to manipulate the forceps to hinge open/closed or slide open/close under selectable finger pressure.

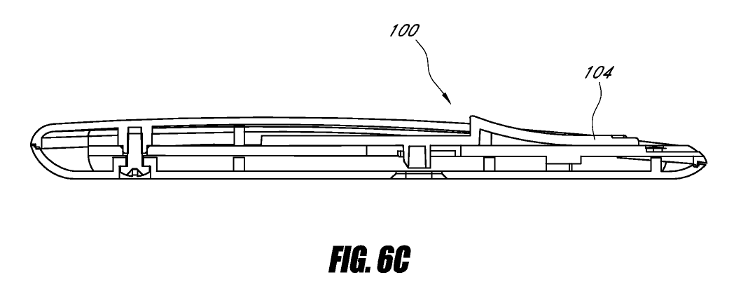

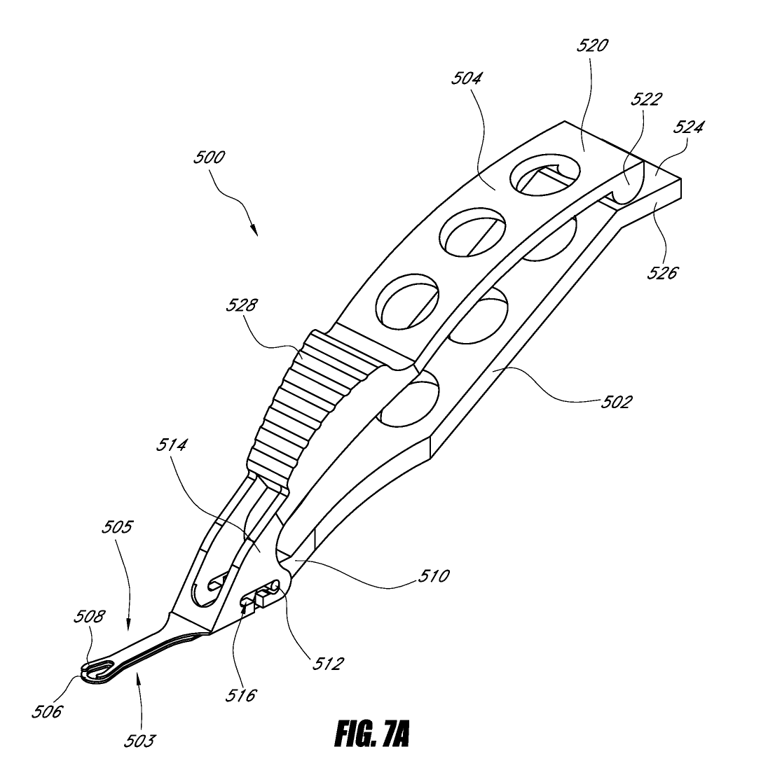

In certain embodiments, the handling tool is a forceps. In a preferred embodiment, the forceps 500 are constructed of a two-component assembly comprising a first arm member (e.g., a fixed component) 502 and a second arm member (e.g., a moveable component) 504, as illustrated in FIG. 7A. The first arm member 502 includes a lower jaw (e.g., first jaw member) 503. In certain embodiments, the lower jaw 503 includes a ring-shaped distal end 506. Preferably, the ring outer diameter is slightly smaller than that of an inlay and more preferably between 60 and 80% of the outer diameter of the inlay. The second arm member 504 includes an upper jaw (e.g., second jaw member) 505. In certain embodiments, the upper jaw 505 includes a partial ring shaped distal end 508. In other embodiments, the distal end 508 of the upper jaw 505 is ring-shaped. For example, the distal end 508 of the upper jaw 505 and the distal end 506 of the lower jaw 503 can both be ring shaped. In certain embodiments, the distal end 508 of the upper jaw 505 and the distal end 506 of the lower jaw 503 are substantially similar (e.g., substantially similar shape and/or size). In some embodiments, the partial ring outer diameter may be equal or smaller than the lower ring or is configured to cover less of the inlay.

The first arm member 502 and the second arm member 504 can be coupled together to provide vertical separation and horizontal translation of the lower jaw 503 and the upper jaw 505 relative to one another. The first arm member 502 can be configured to include a region of relatively straight-sided outer walls 510 over a portion of its length. In certain embodiments, a pin-shaped feature 512 protrudes from both sides of the first arm member 502, the axis of the pin 512 being roughly transverse to the length of the component 502 and perpendicular to the axis of the distal ring. The second arm member 504 can include a region of relatively straight-sided inner walls 514 over a portion of its length. The distance between the inner walls 514 can be slightly greater than the width of the first arm member 502 outer walls 510. A slot 516 (e.g., T-shaped slot) can be cut into both walls 510 of the second arm member 504 at a location within the straight-sided region.

The two components may be assembled in such a way that the straight-walled portion of the second arm member 504 straddles the straight-sided portion of the first arm member 502. The components may be further maneuvered so the T-shaped slots 516 of the second arm member 504 engage with the pin-shaped feature 512 of the first arm member 502. Furthermore, the pins 512 of the first arm member 502 can be receivable within the slots 516 of the second arm member 504 to movably couple the first arm member 502 and the second arm member 504 together. Given the geometry inherent in the components, the second arm member 504 may move relative to the first arm member 502 in either rotation or translation. For example, the second jaw member 505 can be able to at least partially rotate relative to the first jaw member 503 about the pins 512 to provide the vertical separation, and the pins 512 of the first arm member 502 can be able to move within the slot 516 of the second arm member 504 to provide the horizontal translation of the second jaw member 505 relative to the first jaw member 503.

Rotation between the components changes the angle of the jaws relative to one another thereby opening or closing the gap between the jaws in an angular fashion. In a first position, the upper jaw 505 can form an angle relative to the lower jaw 503, e.g., an angle of a few degrees. In certain embodiments, the center of the upper ring resides a fraction of a millimeter to a millimeter above the center of the lower ring.

In a second position, the upper jaw 505 and lower jaw 503 can be hinged toward one another until they meet, e.g., the space between them being nearly zero. In this second position the rings may be roughly parallel, and the axes of the two rings may be approximately coincident. In this second position, the two rings may grasp the inlay on its upper and lower surfaces. The precision jaws may be used to grip a very thin inlay, even if the inlay is as little as 5 microns in thickness.

Beginning in the second position described above, the upper jaw 505 may be translated proximally to uncover the inlay and be in a release position. FIG. 7B illustrates the forceps 500 in the release position where the second arm member 504 has been translated proximally relative to the first arm member 502. The relative position of the pin 512 within the slot 516 has moved from a first end of the slot 516 to a second end of the slot 516. The ability to translate can be a key feature of some embodiments of the forceps 500. Once the inlay is uncovered, it may be lifted into contact with the underside of the pocket or the stromal bed under a flap where it may be readily induced to adhere due to moisture content of the tissue and relative stickiness compared to the jaws of the forceps, as illustrated in FIG. 5B. The jaws may then be withdrawn with confidence that the inlay will remain in place.

The forceps may further include a return spring. Normally, hand instruments utilize a coil spring or leaf spring to provide force to maintain a normally open or normally closed position. A beam spring 518 comprising the proximal extension 520 of the moveable component 504 may serve this function. The far proximal end of the second arm member 504 may terminate in a downward facing bump 522. The bump 522 in turn may rest on a roughly horizontal platen 524 formed at the far proximal end 526 of the fixed component 502. In a preferred embodiment, the first arm member 502 may be relatively rigid while the second arm member 504 may be constructed of a measured spring rate. The spring rate and amount of interference between the bump 522 and the platen 524 results in a net force opposing opening of the jaws when operated in a hinge fashion. Sliding friction between the bump 522 and platen 524 may at least partially and in some embodiments substantially determine opening force when the second arm member 504 is operated in a sliding fashion.

A further embodiment includes a method of joining the parts together. Normally, hand instruments utilize rivets, screws or other fastening methods to secure the first and second arm members and springs into a usable assembly. In certain embodiments, the second arm member 504 mates with the first arm member 502 without the use of additional components. The T-slot 516 fits over the pin 512 as discussed above. Forces developed between the platen 524 and spring 520 provide opposing forces reacted by the pin 512 as it rests against a surface of the T-slot 516 thus holding the two components together. Between procedures, the two components may be separated by hand for autoclave sterilization, then later reassembled for repeated use.

In certain embodiments, the second arm member 504 includes a finger grip 528 capable of providing either hinged or sliding opening motion as desired by the user. The finger grip 528 may be located on a portion of the second arm member 504 proximal to the hinge pin 512. To achieve a hinged opening, the user presses down on the finger grip 528 resulting in an opposing upward motion of the distal tip 508. If the user desires to open the second jaw member 505 in a sliding motion, the user presses horizontally drawing back the finger grip 528 in a direction away from the distal tip 506. This causes the second arm member 504 to move horizontally relative to the first arm member 502. Spring forces cause the hinged motion to return to a closed position, but allow the sliding motion to remain where placed along the length of the T-slot 516.

### Jaw Member Embodiments

Corneal inlays can be very thin (e.g. less than 10 microns thick), and unique inlay handling tools can be used to facilitate insertion of the corneal inlay into an eye. After a corneal pocket or flap is created (e.g., mechanically or with a femtosecond laser), the corneal inlay can be inserted into the corneal pocket or on the stromal bed under a flap with the handling tool (e.g. forceps). In certain embodiments, the handling tool is reusable (e.g., autoclavable). FIGS. 5A-B illustrate insertion of an inlay into a corneal pocket of an eye.

Making the corneal pocket as small as possible is desirable to minimize surgical trauma to the eye and enhance quick post-operative recovery. Typically the pocket entrance is an opening of approximately 4.5 to 5.0 mm. The distal ends (e.g., tips or paddles) of the jaw members, therefore, typically fit into a 4.5 mm slit and are able to open up enough inside the pocket to allow the inlay to be left behind at a location the surgeon selects. The cross-sectional thickness, including height and width, of the distal ends of the jaw members can be small enough to be inserted into the pocket opening and to be maneuvered inside the pocket to deliver the inlay at a precise location.

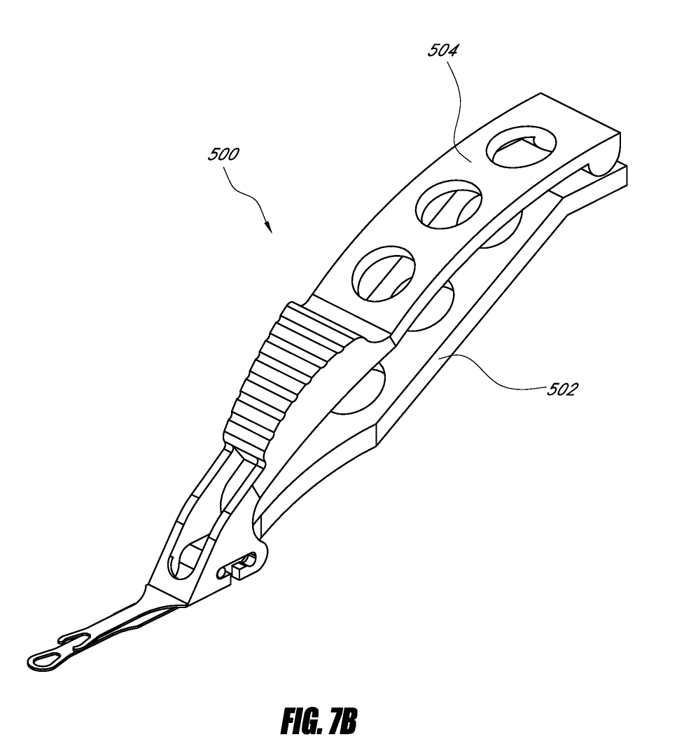

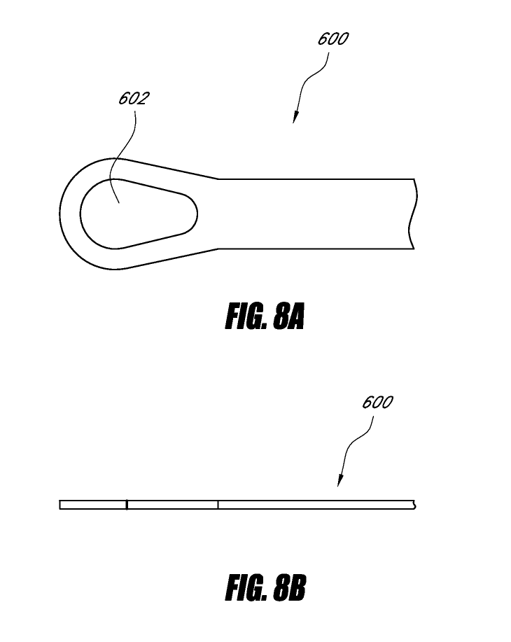

FIGS. 8A-B illustrate one embodiment of a jaw member 600 of an inlay handling tool. In some embodiments, the jaw member has a thickness of about 0.20 mm. In certain embodiments, the distal ends of the jaw members of the inlay handling tool each have substantially the same shape and dimensions as each other. In a certain embodiment, the distal end is a pear shape wherein an outer end is wider than another portion of the jaw member. The pear shape can provide atraumatic interaction with the eye. For example, the distal end can have a portion with a width of about 2.70 mm while another portion of the jaw member has a width of about 1.70 mm. In another example, the distal end can have a portion with width of about 2.60 mm while another portion of the jaw member has a width of about 1.50 mm. In certain embodiments, the outer end of the distal end has a radius of curvature. For example, the radius of curvature of the outer end can be about 1.35 mm or about 1.30 mm.

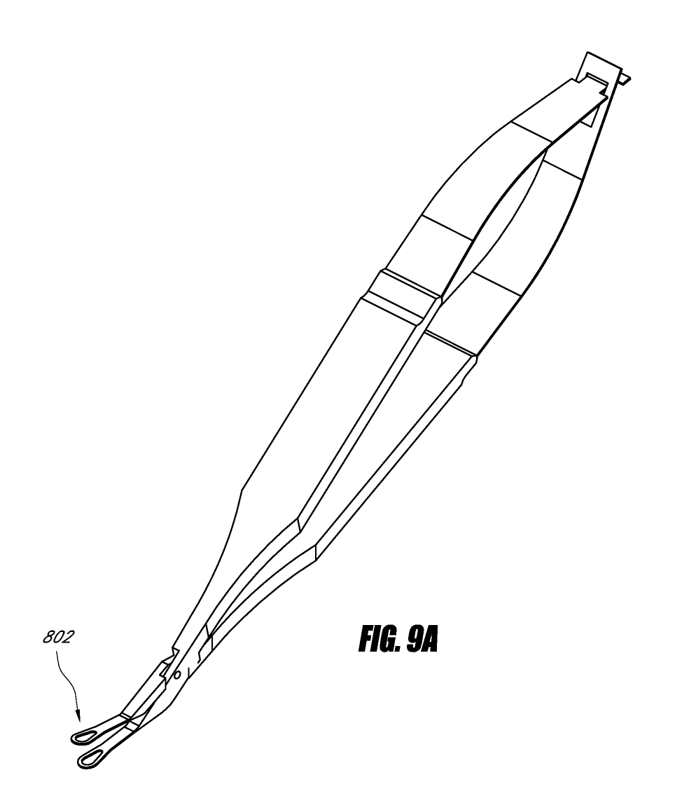

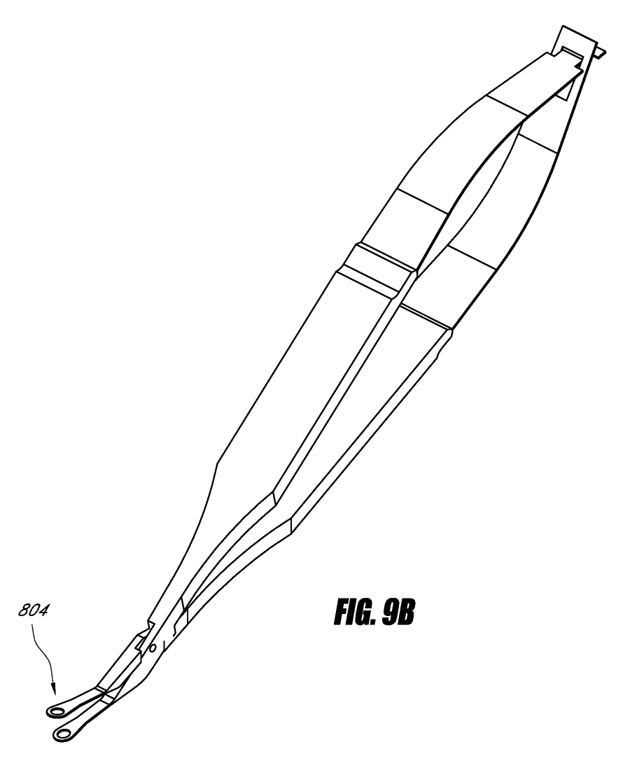

The distal end can have an opening 602 to allow a surgeon to see through the jaw member and through the inlay to center the inlay in the cornea. In certain embodiments, the opening is larger than an inlay opening and smaller than an outer perimeter of the inlay. For example, the opening can have an area about 100% to about 200% of the area of inlay opening. As illustrated in FIGS. 9A-B, the opening in the distal end can be pear shaped 802 or circular 804, respectively. For example, a circular opening can have a diameter of about 1.70 mm. The pear shape can, in certain embodiments, provide additional area for the surgeon to see through compared to circular opening when the distal end is also pear shaped. In further embodiments, additional area can provide additional ability to use an instrument (e.g., hook or spatula) to extend through the hole and make adjustments to the position of the inlay between the jaw members. For example, if the inlay is not correctly positioned between the jaw members, an instrument can be inserted in the hole and used to slide the inlay into a correct position. The pear shape opening preferably has the larger end of the pear shape closer to the outer end of the distal end than the smaller end of the pear shape. Furthermore, the larger end and/or the smaller end of the pear shape can have a radius of curvature. For example, the larger end of the opening can have a radius of curvature of about 0.85 mm or about 0.80 mm. The smaller end of the opening can have a radius of curvature of about 0.425 mm or about 0.43 mm. The width of material between the outer end of the distal end and the larger end of the opening can be about 0.50 mm. In certain embodiments, the distance between the center of the radius of curvature for the larger end and the center of the radius of curvature for the smaller end is about 2.0 mm.

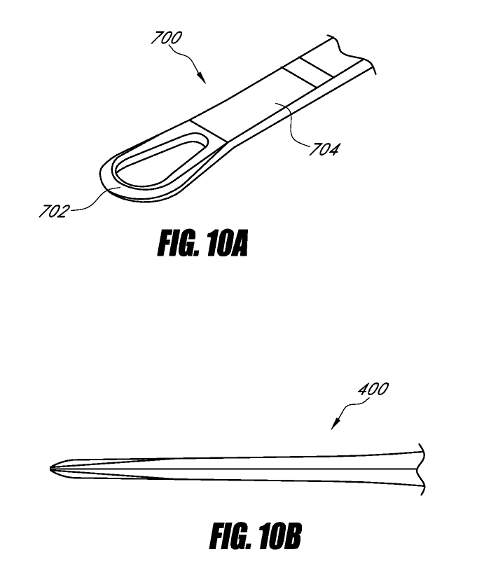

FIGS. 10A-C illustrate another embodiment of a jaw member 700 of a distal end that has beveled paddle edges 702 for smoother transition to allow the distal end to more easily go in and out of a corneal pocket. Differences between the jaw members of FIGS. 8A-B and FIGS. 10A-C are that, for the jaw member 700 of FIGS. 10A-C, the diameter of the tip is 10 microns smaller and the width of the stem of the arm member 704 is 200 microns thinner than for the jaw member 600 of FIGS. 8A-C. Furthermore, the thickness of the jaw members can taper at the distal end. For example, the thickness can be about 0.70 mm at about 4.00 mm from the outer end of the distal end. The outer end of the distal end can have a thickness of about 0.4 mm, and there can be a gradual change in thickness between the outer end to about 4.00 mm from the outer end. In certain embodiments, the material for the jaw member and/or distal end is medical grade stainless steel or medical grade titanium.

In certain embodiments, the corneal inlay, when supported by the jaw member, extends beyond an outer perimeter of the jaw member. As described in the previous section, the outer perimeter of the distal end can be smaller than the diameter of the inlay, for example, between 60 and 80% of the diameter of the inlay. When the corneal inlay extends beyond the jaw member, release and placement of the inlay are facilitated because of the contact between the corneal inlay and the corneal stroma. The jaw member can include a stem that the distal end is attached to. In certain embodiments, at least a portion of the stem neighboring the distal end has a width less than a maximum width of the distal end to help facilitate movement of the inlay within the corneal pocket. The outer perimeter of the distal end can have a smooth curvature to provide atraumatic interaction with the eye. For example, jaw member can have a gradual transition of width between the maximum width of the distal end to the stem. In certain embodiments, the jaw member has a pear shape.

Various other configurations of inlay handling tools are possible. For example, the jaw member can be angled relative to a handle portion of the handling tool. In certain embodiments, the length of the jaw member from the distal end to the start of the angle is about 9.5 mm. In further embodiments, the length of the jaw member from the center of the radius of curvature of the outer end to the start of the angle is about 8.15 mm. In certain embodiments, an angle between the jaw member and a line parallel the handle portion is between about 15 to 45 degrees. Preferably, the angle between the jaw member and a line parallel the handle portion is about 15 degrees. Furthermore, the handling tool can be in a scissors configuration or a tweezers configuration. The handles of the handling tool can be a variety of configurations. For example, the handles can have a textured grip. One particular example of handles is Katena Products, Inc., product number K5-8500.

It should be apparent to those skilled in the art that many combinations and modifications are implicit in the invention. For example, the spring forces could be used to return the second arm member to a closed position in the horizontal sliding direction. Additionally, the hinge spring force could be configured to be normally open instead of normally closed. Additionally, the upper jaw and/or the lower jaw can be configured as a complete circle or a partial circle. The distal ends of the upper and lower jaws disclosed herein can be interchanged with any of the forceps and also jaw members (e.g., upper and lower jaws) disclosed. Furthermore, the lower jaw could be moveable, configured with a finger grip, and made to slide relative to the fixed upper jaw. In the fixed upper jaw scenario, the inlay would favor adhering to the stromal bed instead of the underside of the pocket. Many other combinations and embodiments may readily be applied as dictated by conditions and preferences.

What is claimed is:

1. A loader for use with a corneal inlay comprising:
a platform having a recess configured to receive a corneal inlay;
a cover member slideably contacting the platform, wherein in a closed position, the cover member at least partially covers the recess, and wherein in an open position, the cover member substantially does not cover the recess.

2. The loader of claim 1, wherein the platform further comprises a channel extending from the recess to an exposed surface of the platform and configured to receive a handling tool.

3. The loader of claim 2, further comprising a retainer within the recess configured to restrict the corneal inlay from moving to a bottom of the recess.

4. The loader of claim 3, wherein the retainer is a shelf between a top and a bottom of the recess.

5. The loader of claim 4, wherein a first portion of the recess between the shelf and the bottom of the recess is configured to receive the handling tool.

6. The loader of claim 1, wherein the cover member comprises a retaining feature that extends at least partially into the recess of the platform when in the closed position to prevent the corneal inlay from escaping from the recess.

7. The loader of claim 6, wherein the platform further comprises one or more slots and the retaining feature of the cover member comprises one or more rails that are receivable into the one or more slots in the platform.

8. The loader of claim 1, wherein the recess has a diameter substantially the same as the corneal inlay.

9. A tray for use with a loader of claim 1 configured to restrict the cover member of the loader from moving into the open position when in a locked configuration and to allow the cover member of the loader to move into the open position when in an unlocked configuration.

10. A tray for use with a loader of claim 1 comprising a locking mechanism, wherein in a locked position, the locking mechanism restricts the cover member from moving into the open position, and in an unlocked position, the locking mechanism allows the cover member to move into the open position.

11. The tray of claim 10, wherein the locking mechanism comprises a protrusion on a surface of the tray, wherein in a locked position, the protrusion extends at least partially into an opening of the loader, and in an unlocked position, the protrusion does not extend into the opening of the loader.

12. An inlay handling tool for gripping a corneal inlay comprising:
a first arm member having a first jaw member disposed at a distal end thereof;
a second arm member having a second jaw member disposed at a distal end thereof;
wherein the first arm member and second arm member are coupled together to provide vertical separation and horizontal translation of the first jaw member and the second jaw member relative to one another.

13. The inlay handling tool of claim 12, further comprising:
pins on opposite sides along a length of the first arm member;
slots on opposite sides along a length of the second arm member;
wherein the pins of the first arm member are receivable within the slots of the second arm member to movably couple the first arm member and the second arm member together, the second jaw member being able to at least partially rotate relative to the first jaw member about the pins to provide the vertical separation, and the pins of the first arm member being able to move within the slot of the second arm member to provide the horizontal translation of the second jaw member relative to the first jaw member.

14. The inlay handling tool of claim 13, wherein the slot of the second arm member further comprises an opening that the pins may pass through so that the first arm member and second arm member can be separated.

15. The inlay handling tool of claim 12, wherein the first arm member and the second arm member are biased together.

16. The inlay handling tool of claim 15, wherein the first arm member further comprises a cantilever spring disposed at a proximal end thereof, the second arm member further comprises a platen disposed at a proximal end thereof, and wherein the cantilever spring provides a force on the platen to bias the first jaw member and the second jaw member toward one another.

17. The inlay handling tool of claim 12, wherein the first jaw member comprises a ring and the second jaw member comprises a partial ring.

18. A method of using a corneal inlay delivery system comprising:
providing a loader having a corneal inlay disposed therein, the loader comprising a surface for supporting the corneal inlay and a cover member at least partially covering the corneal inlay;
sliding the cover member to an open position;
inserting a portion of an inlay handling tool beneath the inlay;
gripping the corneal inlay with the inlay handling tool; and
removing the corneal inlay from the loader.

19. The method of claim 18, further comprising disengaging a cover member lock that prevents the cover member from sliding into the open position.

20. The method of claim 18, further comprising inserting the inlay handling tool through a channel extending from the recess to an exposed surface of the platform.

21. The method of claim 18, further comprising:
inserting at least a portion of the inlay handling tool with the corneal inlay into a pocket in a cornea of a patient;
horizontally traversing a jaw member of the inlay handling tool to expose a side of the corneal inlay;
adhering the exposed side of the corneal inlay to an internal wall of the pocket; and
removing the corneal inlay handling tool from the pocket.

22. The method of claim 18, further comprising:
positioning at least a portion of the inlay handling tool with the corneal inlay onto a stroma surface in a cornea of a patient;
horizontally traversing a jaw member of the inlay handling tool to expose a side of the corneal inlay;
adhering the exposed side of the corneal inlay to the stroma surface; and
removing the corneal inlay handling tool away from the stroma surface.
